(19) United States
(12) Patent Application Publication
(10) Pub. No.: US 2025/0177026 A1
(43) Pub. Date: Jun. 5, 2025

(54) ELECTROSURGICAL APPARATUS FOR SEVERING GASTROINTESTINAL TISSUE

(71) Applicant: Covidien LP, Mansfield, MA (US)

(72) Inventors: Ellie K. Ahlquist, San Jose, CA (US); Hillary K. Huszar, Redwood City, CA (US); Ryan W. Bertwell, San Jose, CA (US); Shawn C. Daniel, Mountlake Terrace, WA (US); William J. Camisa, Santa Clara, CA (US)

(21) Appl. No.: 18/841,442

(22) PCT Filed: Feb. 7, 2023
(86) PCT No.: PCT/US2023/012464

§ 371 Date: Aug. 26, 2024

Related U.S. Application Data
(60) Provisional application No. 63/323,560, filed on Mar. 25, 2022.

(51) Int. Cl. A61B 18/00

(52) U.S. Cl. CPC A61B 18/00; A61B 2018/00083; A61B 2018/00101; A61B 2018/00494; A61B 2018/00601; A61B 2018/00982

(57) ABSTRACT

An electrosurgical apparatus for electrosurgically cutting gastrointestinal tissue includes an elongated electrosurgical knife having a radial electrode and a protective tip fabricated from insulative material. The protective tip is axially spaced from the radial electrode to define a gap between the protective tip and the radial electrode.

### CROSS-REFERENCE TO RELATED APPLICATIONS

This application claims the benefit of the filing date of provisional U.S. Patent Application No. 63/323,560, filed on Mar. 25, 2022.

### FIELD

The present technology is related generally to an apparatus for endoscopically severing gastrointestinal tissue.

### BACKGROUND

Endoscopic gastrointestinal procedures, such as endoscopic submucosal dissection (ESD) and endoscopic mucosal resection (EMR), have been accepted as a first choice of the treatment for early stage GI carcinomas because of less invasiveness and lower cost. ESD, for example, allows for an en bloc resection and accurate histopathological diagnosis regardless of the size and location of a lesion or an existence of severe fibrosis at the submucosal layer. During said procedures, surgical tools are utilized for cutting the submucosa.

It would be advantageous to provide a surgical tool that effectively cuts submucosa without damaging adjacent tissue.

### SUMMARY

In accordance with an aspect of the disclosure, an electrosurgical apparatus for severing gastrointestinal tissue is provided. The electrosurgical apparatus may be configured to be hand-held or coupleable to a surgical robotic arm of a surgical robotic system. The electrosurgical apparatus includes an elongated electrosurgical knife configured to couple to a source of electrosurgical energy, and an insulated head coupled to the electrosurgical knife. The electrosurgical knife includes an elongated electrode or portion, and a radial electrode projecting radially outward from the elongated portion. The radial electrode is spaced proximally from the insulated head such that a gap (e.g., an air gap) is defined between the radial electrode and the insulated head.

In aspects, the insulated head may have a proximally-facing surface, and the radial electrode may have a distally-facing surface that is spaced proximally from and facing the proximally-facing surface of the insulated head.

In aspects, the proximally-facing surface of the insulated head and the distally-facing surface of the radial electrode may be parallel with one another.

In aspects, the electrosurgical knife may have a distal end portion axially restrained in the insulated head such that an axial distance of the gap is fixed.

In aspects, the distal end portion of the electrosurgical knife may have a distal flange, and a proximal flange. The insulated head may have an internal collar captured between the distal and proximal flanges.

In aspects, the insulated head may have an internal wall defining an internal cavity of the insulated head. The radial electrode may be axially aligned with the internal cavity.

In aspects, the internal cavity may have a first diameter, and the radial electrode may have a second diameter that is less than the first diameter of the internal cavity.

In aspects, the radial electrode may have an outer peripheral surface disposed radially inward of the internal wall of the insulated head.

In aspects, the electrosurgical knife may be fabricated from metal, and the insulated head may be fabricated from at least one of zirconia or ceramic.

In aspects, the insulated head may have a dome-shaped distal end portion and a cylindrical proximal end portion. The radial electrode may be disc-shaped.

In accordance with another aspect of the present disclosure, an electrosurgical apparatus for severing gastrointestinal tissue is provided that includes an elongated shaft configured for passage through an endoscope, an elongated electrosurgical knife, and an insulated head. The electrosurgical knife includes a rod-shaped portion and a radial electrode projecting radially outward from the rod-shaped portion. The rod-shaped portion has a proximal end portion extending distally from the elongated shaft and configured to couple to a source of electrosurgical energy. The insulated head is coupled to and covers a distal end portion of the rod-shaped portion. The radial electrode is spaced proximally from the insulated head such that an air gap is defined between the radial electrode and the insulated head.

In aspects, the distal end portion of the rod-shaped portion is axially restrained in the insulated head such that an axial distance of the air gap is fixed.

In aspects, the distal end portion of the rod-shaped portion may have a distal flange, and a proximal flange. The insulated head may have an internal collar captured between the distal and proximal flanges.

### BRIEF DESCRIPTION OF THE DRAWINGS

The accompanying drawings, which are incorporated in and constitute a part of the specification, illustrate embodiments of the disclosure and, together with a general description of the disclosure given above as well as the detailed description of the embodiment or embodiments given below, serve to explain the principles of this disclosure.

FIG. 1 is a perspective view illustrating a hand-held electrosurgical apparatus in accordance with an aspect of the present disclosure;

FIG. 2 is a side view illustrating an electrosurgical knife and an insulated head of the electrosurgical apparatus of FIG. 1; and

FIG. 3 is an enlarged view of the electrosurgical knife and the insulated head of FIG. 2.

### DETAILED DESCRIPTION

As used herein, the term “distal” refers to the portion that is being described which is further from a clinician, while the term “proximal” refers to the portion that is being described which is closer to a clinician. As used herein, the terms parallel and perpendicular are understood to include relative configurations that are substantially parallel and substantially perpendicular up to about +/−10 degrees from true parallel and true perpendicular. Further, to the extent consistent, any of the aspects described herein may be used in conjunction with any or all of the other aspects described herein.

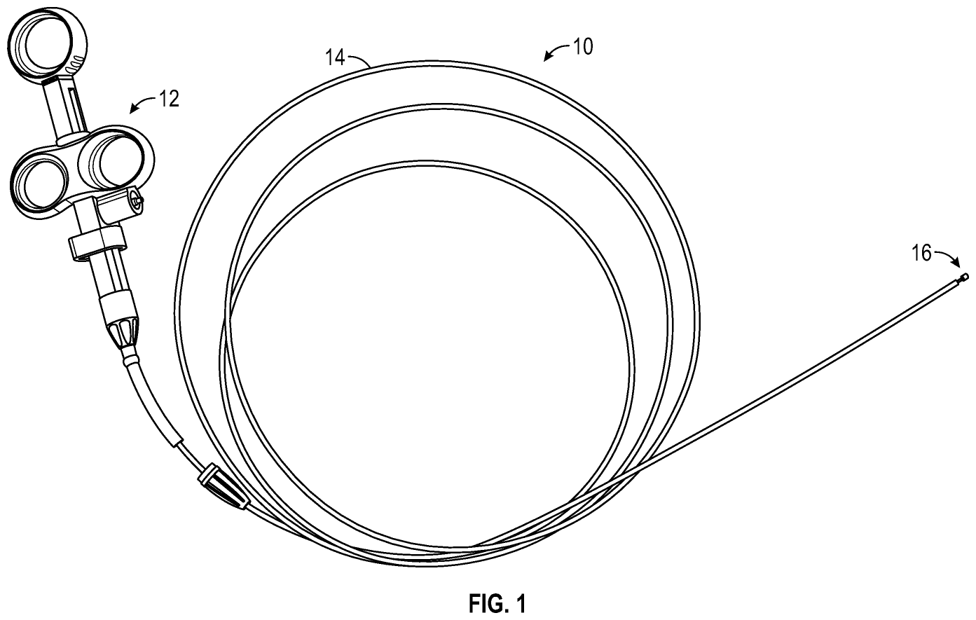

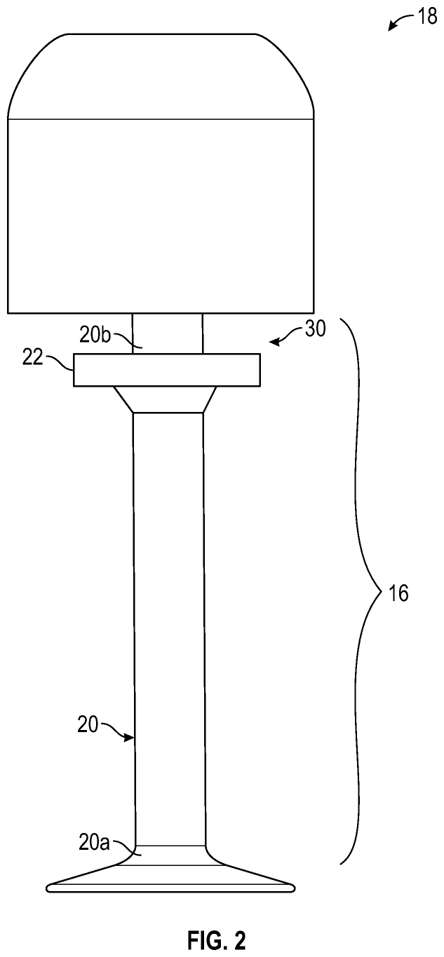

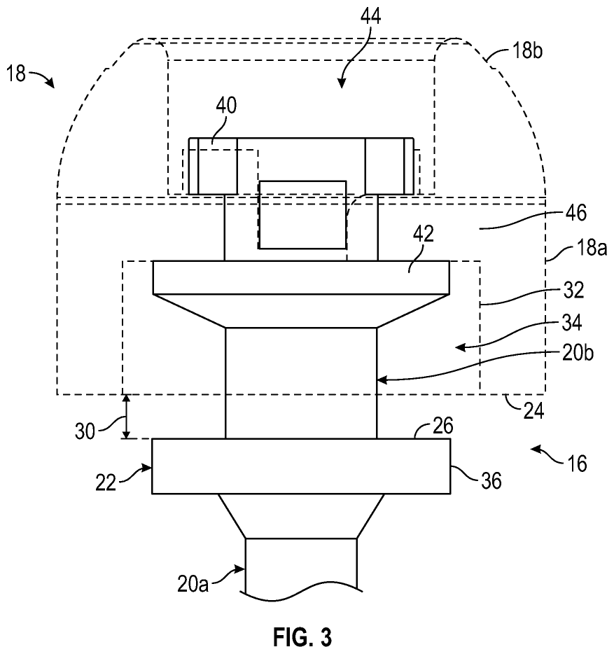

FIG. 1 illustrates a hand-held electrosurgical apparatus 10 configured for passage through a working channel of an endoscope (not shown). The electrosurgical apparatus 10 has a handle assembly 12 to allow for manual actuation of the apparatus 10. However, it is contemplated that the handle assembly 12 may be detachable to allow for coupling of the electrosurgical apparatus 10 to a surgical robotic arm (not shown) of a surgical robotic system for teleoperation of the electrosurgical apparatus 10. The electrosurgical apparatus 10 generally includes an elongated hollow shaft 14 extending distally from the handle assembly 12, an electrosurgical knife 16 extending through the hollow shaft 14, and an insulated head 18 (FIGS. 2-3) coupled to a distal end portion of the electrosurgical knife 16. The electrosurgical knife 16 receives power from an electrosurgical generator (not shown) or other suitable power source (e.g., an internal or external battery) to apply electrosurgical energy (e.g., electrosurgical RF energy or MW energy) to sever tissue that contacts the electrosurgical knife 16. A return pad (not shown) may be placed generally on a patient and provides a return path to the opposite terminal of the generator.

With reference to FIGS. 2-3, the electrosurgical knife 16 is fabricated from a metal (e.g., stainless steel) and includes an elongated portion (e.g., rod-shaped) or electrode 20, and a radial electrode 22 formed with or otherwise coupled to the rod-shaped portion 20 of the knife 16. The rod-shaped portion 20 of the knife 16 has a proximal end portion 20a extending distally from the elongated shaft 14 (FIG. 1) and configured to couple to the source of electrosurgical energy (e.g., via a wire or wires), and a distal end portion 20b (FIG. 3). In aspects, the rod-shaped portion 20 may be axially movable relative to and within the hollow shaft 14 to selectively retract or extend the knife 16 from the hollow shaft 14. The radial electrode 22 may be disc-shaped and project radially outward from the rod-shaped portion 20. Other shapes for the radial electrode 22 are also contemplated. In aspects, the rod-shaped portion 20 may define a channel (not explicitly shown) therethrough configured for passage of an auxiliary electrode (not shown).

In aspects, the electrosurgical apparatus 10 may further include an auxiliary knife electrode (not explicitly shown) that extends through a channel (not explicitly shown) defined longitudinally through the electrosurgical knife 16 and terminates in a distal end configured to electrosurgically cut tissue. The auxiliary knife electrode may be axially movable relative to and through the electrosurgical knife 16 to selectively extend from or retract into a distal end of the insulated head 18. In aspects, the auxiliary knife electrode may define an injection channel longitudinally therethrough configured for the passage of saline or liquid medicine. The injection channel may be open at the distal end of the auxiliary knife electrode for ejection therefrom of the saline or liquid medicine and into mucosa.

The insulated head 18 is secured to and covers the distal end portion 20b of the rod-shaped portion 20 of the knife 16. The insulated head 18 is fabricated from insulative material, such as, for example, ceramic and/or zirconia to insulate the distal end portion 20b of the rod-shaped portion 20 from surrounding tissue during a surgical procedure. The insulated head 18 has a cylindrically-shaped proximal body portion 18a, and a dome-shaped distal end portion 18b. The proximal body portion 18a of the insulated head 18 has a planar, proximally-facing surface 24 that faces a planar, distally-facing surface 26 of the radial electrode 22. The proximally-facing surface 24 may be the proximal-most surface of the insulated head 18, and the distally-facing surface 26 of the radial electrode 22 may be the distal-most surface of the radial electrode 22. The proximally-facing surface 24 of the insulated head 18 may be parallel with the distally-facing surface 26 of the radial electrode 22. The insulated head 18 is positioned distally and longitudinally spaced from the radial electrode 22 such that the distally-facing surface 26 of the radial electrode 22 is spaced proximally from and facing the proximally-facing surface 24 of the insulated head 18 to define a gap, such as, for example, an air gap 30 therebetween. As such, the proximally-facing surface 24 is distal to the distally-facing surface 26. The air gap 30 may be about from about 0.10 mm to about 0.3 mm, and in embodiments about 0.2 mm. The air gap 30 allows for effective electrosurgical cutting of tissue that contacts the radial electrode 22 while still protecting adjacent tissue from inadvertently contacting the radial electrode 22.

The insulated head 18 has an internal wall 32 that defines an internal cavity 34 in the proximal body portion 18a of the insulated head 18. The radial electrode 22 is centrally aligned with the internal cavity 34 along a central longitudinal axis defined by the electrosurgical knife 16. The internal cavity 34 may have a diameter that is greater than a diameter of the radial electrode 22 such that an outer peripheral surface 36 of the radial electrode 22 is disposed radially inward of the internal wall 32 of the insulated head 18.

The distal end portion 20b of the rod-shaped portion 20 of the knife 16 is axially restrained within the insulated head 18 such that an axial distance of the air gap 30 is fixed. More specifically, the distal end portion 20b of the rod-shaped portion 20 has a distal flange 40 and a proximal flange 42, and the distal body portion 18b of the insulated head 18 has an internal cavity 44 separated from the internal cavity 34 of the proximal body portion 18a by an internal collar 46 of the insulated head 18. The internal collar 46 has a thickness identical to or substantially identical to the axial distance between the distal and proximal flanges 40, 42. The internal collar 46 of the insulated head 18 is captured between the distal and proximal flanges 40, 42 to fix the insulated head 18 to the knife 16. Additionally or alternately, the insulated head 18 may be secured to the distal end portion of the knife 16 by adhesive, welding, or the like.

In use, during an endoscopic resection, such as ESD, lesions in the mucosal and submucosal space need to be removed. The hollow shaft 14 of the electrosurgical apparatus 10 is passed through a working channel of an endoscope to position the electrosurgical knife 16 adjacent the lesion. The electrosurgical knife 16 is activated to apply electrosurgical energy from the outer peripheral surface 36 of the radial electrode 22 to the lesion to electrosurgically sever the lesion. The insulated head 18 prevents surrounding tissue from being severed by the radial electrode 22. In addition to the outer peripheral edge 36 of the radial electrode 22 contacting and severing tissue, a portion of the tissue may enter the air gap 30 between the insulated head 18 and the radial electrode 22 to contact the proximally-oriented surface 24 of the radial electrode 22 whereby more efficient cutting of the tissue occurs.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

What is claimed is:

1. An electrosurgical apparatus for severing gastrointestinal tissue, the electrosurgical apparatus comprising:
an elongated electrosurgical knife configured to couple to a source of electrosurgical energy and including:
an elongated portion; and
a radial electrode projecting radially outward from the elongated portion; and
an insulated head coupled to the elongated electrosurgical knife, wherein the radial electrode is spaced proximally from the insulated head such that a gap is defined between the radial electrode and the insulated head.

2. The electrosurgical apparatus according to claim 1, wherein the insulated head has a proximally-facing surface, and the radial electrode has a distally-facing surface that is spaced proximally from and facing the proximally-facing surface of the insulated head.

3. The electrosurgical apparatus according to claim 2, wherein the proximally-facing surface of the insulated head and the distally-facing surface of the radial electrode are parallel with one another.

4. The electrosurgical apparatus according to claim 1, wherein the elongated electrosurgical knife has a distal end portion axially restrained in the insulated head such that an axial distance of the gap is fixed.

5. The electrosurgical apparatus according to claim 4, wherein the distal end portion of the elongated electrosurgical knife has a distal flange, and a proximal flange, the insulated head having an internal collar captured between the distal and proximal flanges.

6. The electrosurgical apparatus according to claim 1, wherein the insulated head has an internal wall defining an internal cavity of the insulated head, the radial electrode being axially aligned with the internal cavity.

7. The electrosurgical apparatus according to claim 6, wherein the internal cavity has a first diameter, and the radial electrode has a second diameter that is less than the first diameter of the internal cavity.

8. The electrosurgical apparatus according to claim 7, wherein the radial electrode has an outer peripheral surface disposed radially inward of the internal wall of the insulated head.

9. The electrosurgical apparatus according to claim 1, wherein the elongated electrosurgical knife is fabricated from metal, and the insulated head is fabricated from at least one of zirconia or ceramic.

10. The electrosurgical apparatus according to claim 1, wherein the insulated head has a dome-shaped distal end portion and a cylindrical proximal end portion, and the radial electrode is disc-shaped.

11. An electrosurgical apparatus for severing gastrointestinal tissue, the electrosurgical apparatus comprising:
an elongated shaft configured for passage through an endoscope;
an elongated electrosurgical knife including:
a rod-shaped portion having a proximal end portion extending distally from the elongated shaft and configured to couple to a source of electrosurgical energy, and a distal end portion; and
a radial electrode projecting radially outward from the rod-shaped portion; and
an insulated head coupled to and covering the distal end portion of the rod-shaped portion, wherein the radial electrode is spaced proximally from the insulated head such that an air gap is defined between the radial electrode and the insulated head.

12. The electrosurgical apparatus according to claim 11, wherein the insulated head has a proximally-facing surface, and the radial electrode has a distally-facing surface that is spaced proximally from and facing the proximally-facing surface of the insulated head.

13. The electrosurgical apparatus according to claim 12, wherein the proximally-facing surface of the insulated head and the distally-facing surface of the radial electrode are parallel with one another.

14. The electrosurgical apparatus according to claim 11, wherein the distal end portion of the rod-shaped portion is axially restrained in the insulated head such that an axial distance of the air gap is fixed.

15. The electrosurgical apparatus according to claim 14, wherein the distal end portion of the rod-shaped portion has a distal flange, and a proximal flange, the insulated head having an internal collar captured between the distal and proximal flanges.

16. The electrosurgical apparatus according to claim 11, wherein the insulated head has an internal wall defining an internal cavity of the insulated head, the radial electrode being axially aligned with the internal cavity.

17. The electrosurgical apparatus according to claim 16, wherein the internal cavity has a first diameter, and the radial electrode has a second diameter that is less than the first diameter of the internal cavity.

18. The electrosurgical apparatus according to claim 16, wherein the radial electrode has an outer peripheral surface disposed radially inward of the internal wall of the insulated head.

19. The electrosurgical apparatus according to claim 11, wherein the elongated electrosurgical knife is fabricated from metal, and the insulated head is fabricated from at least one of zirconia or ceramic.

20. The electrosurgical apparatus according to claim 11, wherein the insulated head has a dome-shaped distal end portion and a cylindrical proximal end portion, and the radial electrode is disc-shaped.
